(19) United States
(12) Patent Application Publication
(10) Pub. No.: US 2024/0376547 A1
(43) Pub. Date: Nov. 14, 2024

(54) ONCOLOGIC VARIATIONS ASSOCIATED WITH CANCER AND METHODS OF TREATMENT

(71) Applicant: LINNAEUS THERAPEUTICS, INC., Haddonfield, NJ (US)

(72) Inventors: Tina GARYANTES, Bradley Beach, NJ (US); Patrick MOONEY, Haddonfield, NJ (US); Christopher NATALE, Philadelphia, PA (US)

(21) Appl. No.: 18/554,260

(22) PCT Filed: Apr. 7, 2022
(86) PCT No.: PCT/US2022/023768

§ 371 Date: Oct. 6, 2023

Related U.S. Application Data
(60) Provisional application No. 63/172,485, filed on Apr. 8, 2021.

(51) Int. Cl. C12Q 1/6886; A61K 31/473; A61P 35/00

(52) U.S. Cl. CPC C12Q 1/6886; A61K 31/473; A61P 35/00; C12Q 2600/106; C12Q 2600/156

(57) ABSTRACT

The disclosure provides oncologic variations (i.e., gene fusions, gene mutations and gene amplifications) relating to cancer, as well as methods of treating cancer and identifying patients amenable to treatment.

### BACKGROUND OF THE DISCLOSURE

### Field of the Disclosure

The present disclosure relates generally to detecting oncogenic variations of genomic DNA that are related to cancer, including cancers that are refractory to cancer therapy, and to identifying cancers amenable to treatment with LNS8801.

### Technical Background

Many cancers are characterized by disruptions in cellular signaling pathways that lead to aberrant control of cellular processes, or to uncontrolled growth and proliferation of cells. It is also known that gene mutations, deletions and/or translocations resulting in fusion proteins with aberrant signaling activity can directly lead to certain cancers. For example, the BCR-ABL oncoprotein, a tyrosine kinase fusion protein that is the causative agent in human chronic myelogenous leukemia (CML) and is found in at least 90-95% of CML cases, is generated by the translocation of gene sequences from the c-ABL protein tyrosine kinase on chromosome 9 into BCR sequences on chromosome 22 (Kurzock et al., N. Engl. J. Med. 319: 990-998 (1988)).

As another example, Falini et al. (Blood, 99(2), 409-426 (2002)) and Hallberg et al. (Annals of Oncology, 27(supp. 3); iii4-iii15, (2016)), review translocations known to occur in hematological cancers, including the NPM-ALK fusion found in anaplastic large cell lymphoma (ALCL). The general role of ALK in cancer has been described (Pulford et al., J. Cell Physiology, 199(3), 330-358 (2004); Hallberg, B. et al., Annals of Oncology, 27(supp. 3); iii4-iii5, (2016)), and a key modulatory mechanism, at least in certain cancers, appears to be that ALK regulates transcriptional expression of MYC and activates c-MYC transactivation of c-MYC target genes (Pilling et al., Oncotarget, 9(10), 8823-8835 (2018)), activity shared with BCR-ABL (Xie, et al., Oncogene 21, 7137-48 (2002)) and other oncogenic variations.

Detecting and identifying mutations present in human cancers is highly desirable because such information can guide treatment decisions, explain why a cancer is refractory to certain treatments and even lead to the development of new therapeutics that target such fusion or mutant proteins and to new diagnostics for identifying patients that have such gene mutations.

Accordingly, there remains a need for the detection and/or identification of oncogenic variations, such as translocations or deletions, resulting in fusion or mutant proteins implicated in the formation and progression of cancers, including cancers that are refractory to cancer therapy. Identification of such fusion proteins will, among other things, desirably enable new methods for selecting patients for targeted therapies like LNS8801.

### SUMMARY OF THE DISCLOSURE

One aspect of the disclosure provides a method of treating cancer in a patient in need thereof, comprising obtaining a sample from the patient, analyzing the sample for one or more oncologic variations, determining the patient is amenable to treatment with LNS8801 if one or more oncogenic variations is found, and administering to the patient an effective amount of LNS8801.

Another aspect of the disclosure provides a method of identifying a patient amenable to treatment with LNS8801 therapy, comprising obtaining a sample from the patient, analyzing the sample for one or more oncologic variations, determining the patient is amenable to treatment with LNS8801 if one or more oncogenic variations is found, and administering to the patient an effective amount of LNS8801.

In embodiments of the various aspects of the disclosure, the oncologic variations comprise one or more gene fusions, gene mutations, gene duplications or combinations thereof, which fusions, mutations and duplications affect, e.g., oncogenes, tumor suppressor genes and DNA repair genes.

In embodiments of the various aspects of the disclosure, oncologic variations can confer resistance to one or more cancer therapies (e.g., immune checkpoint therapy agent directed against, e.g., PD-1, PD-1, CTLA4, CD40, OX40, TIGIT, CD137 and targeted inhibitor against, e.g., EGFR, BRAF, MEK, ALK, JAK1/2, VEGF, SRC, BTK, AKT, MTOR, SCL-2, ESR1, FGFR, MET), which, in some embodiments can be driven by an increase in amount or activity of one or more proteins from Myc family genes.

Additional aspects of the disclosure will be evident from the disclosure herein.

### BRIEF DESCRIPTION OF THE DRAWINGS

FIG. 1 presents a graph of results from a proliferation assay of parental and EML4-ALK A549 NSCLC cells treated with LNS8801.

FIG. 2 presents a graph of results from a proliferation assay of Crizotinib naïve or resistant EML4-ALK A549 NSCLC cells treated with LNS8801.

FIG. 3 presents a graph of results from a proliferation assay of Crizotinib naïve or resistant EML4-ALK A549 NSCLC cells treated with Crizotinib. Combined with FIG. 12 and FIG. 13, FIG. 14 demonstrates that ALK+ NSCLC cells are more sensitive to LNS8801 than isogenic controls, and that LNS8801 has activity in the setting of ALK-inhibitor resistance.

### DETAILED DESCRIPTION

The present inventors have unexpectedly determined that certain oncogenic variations not only promote cellular transformation to a cancerous state but are predictive of a positive or refractory response to certain treatments, including LNS8801.

### Definitions

The term “marker” or “biomarker” refers to a molecule (typically protein, nucleic acid, carbohydrate, or lipid) that is expressed in the cell, expressed on the surface of a cancer cell or secreted by a cancer cell in comparison to a non-cancer cell, and which is useful for the diagnosis of cancer, for providing a prognosis, and for preferential targeting of a pharmacological agent to the cancer cell. Such markers are often molecules that are overexpressed in a cancer cell in comparison to a non-cancer cell, for instance, 1-fold overexpression, 2-fold overexpression, 3-fold overexpression or more in comparison to a normal cell. Further, a marker can be a molecule that is inappropriately synthesized in the cancer cell, for instance, a molecule that contains deletions, additions (including amplifications/multiple copies) or mutations in comparison to the molecule expressed on a normal cell. Alternatively, such biomarkers are molecules that are underexpressed in a cancer cell in comparison to a non-cancer cell, for instance, 1-fold underexpression, 2-fold underexpression, 3-fold underexpression, or more. Further, a marker can be a molecule that is inappropriately synthesized in cancer, for instance, a molecule that contains deletions, additions or mutations in comparison to the molecule expressed on a normal cell.

It will be understood by the skilled artisan that markers may be used in combination with other markers or tests for any of the uses, e.g., prediction, diagnosis, or prognosis of cancer, amenability of a cancer to specific treatment and the like, disclosed herein.

“Biological sample” includes a tissue sample, a cell culture, or a bodily fluid. The bodily fluid can be any useful fluid, including without limitation one or more of peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, and umbilical cord blood. In some embodiments, the bodily fluid comprises blood, serum or plasma.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., lung etc.), the size and type of the tumor, among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue from within the tumor. A diagnosis or prognosis made by endoscopy or radiographic guidance can require a “core-needle biopsy”, or a “fine-needle aspiration biopsy” which generally obtains a suspension of cells from within a target tissue. Biopsy techniques are discussed, for example, in Harrison's Principles of Internal Medicine, Kasper, et al., eds., 16th ed., 2005, Chapter 70, and throughout Part V.

The terms “overexpress,” “overexpression,” or “overexpressed” interchangeably refer to a protein or nucleic acid that is translated or transcribed at a detectably greater level, usually in a cancer cell, in comparison to a normal cell. The term includes overexpression due to transcription, post transcriptional processing, translation, post-translational processing, cellular localization (e.g., organelle, cytoplasm, nucleus, cell surface), RNA and protein stability, and presence of an aberrant number of gene copies as compared to a normal cell.

Overexpression can be detected using conventional techniques for detecting DNA and mRNA (i.e., RT-PCR, PCR (including its variants known in the art), hybridization, e.g., in situ hybridization, fluorescence or otherwise) or proteins (i.e., ELISA, immunohistochemical techniques). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “underexpress,” “underexpression,” or “underexpressed” or “downregulated” interchangeably refer to a protein or nucleic acid that is translated or transcribed at a detectably lower level in a cancer cell, in comparison to a normal cell. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., RT-PCR, PCR, hybridization) or proteins (i.e., ELISA, immunohistochemical techniques). Underexpression can be 10%, 20%, 30%, 40%, 50%, 0%, 70%, 80%, 90% or less in comparison to a control. In certain instances, underexpression is 1-fold, 2-fold, 3-fold, 4-fold or more lower levels of transcription or translation in comparison to a control.

The term “differentially expressed” or “differentially regulated” refers generally to a protein or nucleic acid that is overexpressed (upregulated) or underexpressed (downregulated) in one sample compared to at least one other sample, generally in a cancer patient compared to a sample of non-cancerous tissue in the context of the present invention.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serino (S), Threonine (T); and 8) Cysteine (C), Methionine (M). See, e.g., Creighton, Proteins (1984).

The phrase “specifically (or selectively) binds” when referring to a protein, nucleic acid, antibody, or small molecule compound refers to a binding reaction that is determinative of the presence of the protein or nucleic acid, such as the differentially expressed genes of the present invention, often in a heterogeneous population of proteins or nucleic acids and other biologics.

The phrase “functional effects” in the context of assays for testing compounds that modulate a marker protein includes the determination of a parameter that is indirectly or directly under the influence of a biomarker of the invention, e.g., a chemical or phenotypic. A functional effect therefore includes ligand binding activity, transcriptional activation or repression, the ability of cells to proliferate, the ability to migrate, among others. “Functional effects” include in vitro, in vivo, and ex vivo activities.

By “determining the functional effect” is meant assaying for a compound that increases or decreases a parameter that is indirectly or directly under the influence of a biomarker of the invention, e.g., measuring physical and chemical or phenotypic effects. Such functional effects can be measured by any means known to those skilled in the art, e.g., changes in spectroscopic characteristics (e.g., fluorescence, absorbance, refractive index); hydrodynamic (e.g., shape), chromatographic; or solubility properties for the protein; ligand binding assays, e.g., binding to antibodies; measuring inducible markers or transcriptional activation of the marker; measuring changes in enzymatic activity; the ability to Increase or decrease cellular proliferation, apoptosis, cell cycle arrest, measuring changes in cell surface markers. The functional effects can be evaluated by many means known to those skilled in the art, e.g., microscopy for quantitative or qualitative measures of alterations in morphological features, measurement of changes in RNA or protein levels for other genes expressed in placental tissue, measurement of RNA stability, identification of downstream or reporter gene expression (CAT, luciferase, R-gal. GFP and the like), e.g., via chemiluminescence, fluorescence, colorimetric reactions, antibody binding, inducible markers, etc.

“Inhibitors,” “activators.” and “modulators” of the markers are used to refer to activating, inhibitory, or modulating molecules identified using in vitro and in vivo assays of cancer biomarkers. Inhibitors are compounds that, e.g., bind to, partially or totally block activity, decrease, prevent, delay activation, inactivate, desensitize, or down regulate the activity or expression of cancer biomarkers. “Activators” are compounds that Increase, open, activate, facilitate, enhance activation, sensitize, agonize, or up regulate activity of cancer biomarkers, e.g., agonists. Inhibitors, activators, or modulators also include genetically modified versions of cancer biomarkers, e.g., versions with altered activity, as well as naturally occurring and synthetic ligands, antagonists, agonists, antibodies, peptides, cyclic peptides, nucleic acids, antisense molecules, ribozymes, RNAi and siRNA molecules, small organic molecules and the like. Such assays for inhibitors and activators include, e.g., expressing cancer biomarkers in vitro, in cells, or cell extracts, applying putative modulator compounds, and then determining the functional effects on activity, as described above.

A “probe” or “probes” refers to a polynucleotide that is at least eight (8) nucleotides in length and which forms a hybrid structure with a target sequence, due to complementarity of at least one sequence in the probe with a sequence in the target region. The polynucleotide can be composed of DNA and/or RNA. Probes in certain embodiments, are detectably labeled, as discussed in more detail herein. Probes can vary significantly in size. Generally, probes are, for example, at least 8 to 15 nucleotides in length. Other probes are, for example, at least 20, 30 or 40 nucleotides long. Still other probes are somewhat longer, being at least, for example, 50, 60, 70, 80, 90 nucleotides long. Yet other probes are longer still, and are at least, for example, 100, 150, 200 or more nucleotides long. Probes can be of any specific length that falls within the foregoing ranges as well. Preferably, the probe does not contain a sequence complementary to the sequence(s) used to prime for a target sequence during the polymerase chain reaction.

The terms “complementary” or “complementarity” are used in reference to polynucleotides (that is, a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Alternatively, there may be “complete” or “total” complementarity between the nucleic acids. The degree of complementarity between nucleic acid strands has significant effects on the efficiency and strength of hybridization between nucleic acid strands.

“Oligonucleotide” or “polynucleotide” refers to a polymeric form of nucleotides of any length, either deoxyribonucleotide or ribonucleotide. These terms include, but are not limited to, a single-, double- or triple-stranded DNA, genomic DNA, cDNA, RNA, DNA-RNA hybrid, or a polymer comprising purine and pyrimidine bases or other natural chemically, biochemically modified non-natural or derivatized nucleotide bases.

“Amplification detection assay” refers to a primer pair and matched probe wherein the primer pair flanks a region of a target nucleic acid, typically a target gene, that defines an amplicon, and wherein the probe binds to the amplicon.

The terms “oncologic variation”, “genetic variant” and “nucleotide variant” are used herein interchangeably to refer to changes or alterations to the reference human gene or cDNA sequence at a particular locus, including, but not limited to, nucleotide base deletions, insertions, inversions, and substitutions (e.g., gene fusions) in the coding and noncoding regions. Deletions may be of a single nucleotide base, a portion or a region of the nucleotide sequence of the gene, or of the entire gene sequence. Insertions may be of one or more nucleotide bases. The“oncologic variation”, “genetic variant” or“nucleotide variant” may occur in transcriptional regulatory regions, untranslated regions of mRNA, exons, introns, or exon/intron junctions. The “genetic variant” or “nucleotide variant” may or may not result in stop codons, frame shifts, deletions of amino acids, altered gene transcript splice forms or altered amino acid sequence.

The term “gene” refers to a polynucleotide (e.g., a DNA segment), that encodes a polypeptide and includes regions preceding and following the coding regions as well as intervening sequences (introns) between individual coding segments (exons). Parent genes or protein sequences are presented as Entrez Gene IDs or accession numbers. For example, the ALK Entrez Gene ID is 238. If any changes have been made to the sequence in the Gene ID in Entrez, the change is indicated after the Gene ID with a decimal and the number of the change (e.g., 238.1). Further, for example, ALK has the accession number Q9UM73.

As used herein, the term “amino acid variant” is used to refer to an amino acid change to a reference human protein sequence resulting from “genetic variant” or “nucleotide variant” to the reference human gene encoding the reference protein. The term “amino acid variant” is intended to encompass not only single amino acid substitutions, but also amino acid deletions, insertions, and other significant changes of amino acid sequence in the reference protein. Variants of the invention are described by the following nomenclature: [original amino acid residue/position/substituted amino acid residue]. For example, the substitution of leucine for arginine at position 76 is represented as R76L.

The term “genotype” as used herein means the nucleotide characters at a particular nucleotide variant marker (or locus) in either one allele or both alleles of a gene (or a particular chromosome region). With respect to a particular nucleotide position of a gene of interest, the nucleotide(s) at that locus or equivalent thereof in one or both alleles form the genotype of the gene at that locus. A genotype can be homozygous or heterozygous. Accordingly, “genotyping” means determining the genotype, that is, the nucleotide(s) at a particular gene locus. Genotyping can also be done by determining the amino acid variant at a particular position of a protein which can be used to deduce the corresponding nucleotide variant(s).

A set of probes typically refers to a set of primers, usually primer pairs, and/or detectably-labeled probes that are used to detect the target genetic variations. The primer pairs are used in an amplification reaction to define an amplicon that spans a region for a target genetic variation for each of the aforementioned genes. The set of amplicons are detected by a set of matched probes. In an exemplary embodiment, the invention is a set of TaqMan™ (Roche Molecular Systems. Pleasanton, Calif.) assays that are used to detect a set of target genetic variations used in the methods of the invention.

In one embodiment, the set of probes are a set of primers used to generate amplicons that are detected by a nucleic acid sequencing reaction, such as a next generation sequencing reaction. In these embodiments, for example, AmpliSEQ™ (Life Technologies/Ion Torrent, Carlsbad, Calif.) or TruSEQ™ (Illumina, San Diego, Calif.) technology can be employed. In other embodiments, the two or more probes are primer pairs.

“Hybridize” or ‘hybridization’ refers to the binding between nucleic acids. The conditions for hybridization can be varied according to the sequence homology of the nucleic acids to be bound. Thus, if the sequence homology between the subject nucleic acids is high, stringent conditions are used. If the sequence homology is low, mild conditions are used. When the hybridization conditions are stringent, the hybridization specificity increases, and this increase of the hybridization specificity leads to a decrease in the yield of non-specific hybridization products. However, under mild hybridization conditions, the hybridization specificity decreases, and this decrease in the hybridization specificity leads to an increase in the yield of non-specific hybridization products.

“Stringent conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, etc.

Hybridization between nucleic acids can occur between a DNA molecule and a DNA molecule, hybridization between a DNA molecule and a RNA molecule, and hybridization between a RNA molecule and a RNA molecule.

A “mutein” or “variant” refers to a polynucleotide or polypeptide that differs relative to a wild-type or the most prevalent form in a population of individuals by the exchange, deletion, or insertion of one or more nucleotides or amino acids, respectively. The number of nucleotides or amino acids exchanged, deleted, or inserted can be 1, 2, 3, 4, 5, 6, 7, 8, 9, 10, 11, 12, 13, 14, 15, 16, 17, 18, 19, 20 or more such as 25, 30, 35, 40, 45 or 50. The term mutein can also encompass a translocation, for example the fusion of the polypeptides encoded by the ALK and TPM1 genes (TPM1/ALK).

“Gene fusion” refers to a chimeric genomic DNA resulting from the fusion of at least a portion of a first gene to a portion of a second gene. The point of transition between the sequence from the first gene in the fusion to the sequence from the second gene in the fusion is referred to as the “breakpoint” or “fusion point.”

Transcription of the gene fusion results in a chimeric mRNA.

“Single nucleotide polymorphism” or “SNP” refers to a DNA sequence variation that occurs when a single nucleotide (A, T, G, or C) in the genome differs between members of a biological species or paired chromosomes in a human.

“Mutation” is defined herein as a specific change at a genomic location, i.e.: Chromosome, start, stop, reference base, alternate base, variant type (SNP, INS, DEL) etc.

A “primer” or “primer sequence” refers to an oligonucleotide that hybridizes to a target nucleic acid sequence (for example, a DNA template to be amplified) to prime a nucleic acid synthesis reaction. The primer may be a DNA oligonucleotide, a RNA oligonucleotide, or a chimeric sequence. The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3-4 nucleotides long) do not form thermodynamically stable duplexes with target nucleic acids under such hybridization conditions. The upper limit is often determined by the possibility of having a duplex formation in a region other than the pre-determined nucleic acid sequence in the target nucleic acid. Generally, suitable primer lengths are in the range of about 10 to about 40 nucleotides long. In certain embodiments, for example, a primer can be 10-40, 15-30, or 10-20 nucleotides long. A primer is capable of acting as a point of initiation of synthesis on a polynucleotide sequence when placed under appropriate conditions.

The primer will be completely or substantially complementary to a region of the target polynucleotide sequence to be copied. Therefore, under conditions conducive to hybridization, the primer will anneal to the complementary region of the target sequence. Upon addition of suitable reactants, including, but not limited to, a polymerase, nucleotide triphosphates, etc., the primer is extended by the polymerizing agent to form a copy of the target sequence. The primer may be single-stranded or alternatively may be partially double-stranded.

“Detection,” “detectable” and grammatical equivalents thereof refers to ways of determining the presence and/or quantity and/or identity of a target nucleic acid sequence. In some embodiments, detection occurs amplifying the target nucleic acid sequence. In other embodiments, sequencing of the target nucleic acid can be characterized as “detecting” the target nucleic acid. A label attached to the probe can include any of a variety of different labels known in the art that can be detected by, for example, chemical or physical means. Labels that can be attached to probes may include, for example, fluorescent and luminescence materials.

“Amplifying,” “amplification,” and grammatical equivalents thereof refers to any method by which at least a part of a target nucleic acid sequence is reproduced in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridg, UK), and self-sustained sequence replication (3SR), including multiplex versions or combinations thereof, for example but not limited to, OLA/PCR. PCR/OLA, LDR/PCR, PCR/PCR/LDR, PCR/LDR, LCR/PCR, PCR/LCR (also known as combined chain reaction-CCR), and the like. Descriptions of such techniques can be found in, among other places, Sambrook et al. Molecular Cloning, 3rd Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humans Press, Totowa, N.J. (2002).

Analysis of nucleic acid markers can be performed using techniques known in the art including, without limitation, sequence analysis, and electrophoretic analysis. Non-limiting examples of sequence analysis include Maxam-Gilbert sequencing, Sanger sequencing, capillary array DNA sequencing, thermal cycle sequencing (Sears at al., Biotechniques, 13:826-633 (1992)), solid-phase sequencing (Zimmerman et al., Methods Mol. Cell. Biol., 3:39-42 (1992)), sequencing with mass spectrometry such as matrix-assisted laser desorption/ionization time-of-flight mass spectrometry (MALDI-TOF/MS; Fu at al., Nat. Biotechnol., 16:381-384 (1998)), and sequencing by hybridization. Chee at al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MISEQ, and the Roche/454 next generation sequencing system.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

“Detectably labeled probe” or “detector probe” refers to a molecule used in an amplification reaction, typically for quantitative or real-time PCR analysis, as well as end-point analysis. Such detector probes can be used to monitor the amplification of the target nucleic acid sequence. In some embodiments, detector probes present in an amplification reaction are suitable for monitoring the amount of amplicon(s) produced as a function of time. Such detector probes include, but are not limited to, the 5′-exonuclease assay (TAQMAN® probes described herein (see also U.S. Pat. No. 5,538,848) various stem-loop molecular beacons (see for example. U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see. e.g., U.S. Pat. Nos. 6,355,421 and 6,593,091), linear PNA beacons (see, for example, Kubista at al., 2001, SPIE 4264:53-58), non-FRET probes (see, for example, U.S. Pat. No. 8,150,097), Sunrise®/Amplifluor™ probes (U.S. Pat. No. 6,548,250), stem-loop and duplex Scorpion probes (Solinas at al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807: Isacsson et al., 2000. Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093: Zhang et al., 2002 Shanghai. 34:329-332; Maxwell et al., 2002, J. Am. Chem. Soc. 124:9606-9612; Broude at al., 2002, Trends Biotechnol. 20:249-56; Huang et al., 2002, Chem. Res. Toxicol. 15:118-126; and Yu et al., 2001, J. Am. Chem. Soc 14:11155-11161.

Detector probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detector probes can also include two probes, wherein for example a fluor is on one probe, and a quencher is on the other probe, wherein hybridization of the two probes together on a target quenches the signal, or wherein hybridization on the target alters the signal signature via a change in fluorescence. Detector probes can also comprise sulfonate derivatives of fluorescein dyes with SO3 instead of the carboxylate group, phosphoramidite forms of fluorescein, phosphoramidite forms of CYS (commercially available for example from Amersham). In some embodiments, interchelating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, the detector probes of the present teachings have a Tₘ of 63-69° C., though it will be appreciated that guided by the present teachings routine experimentation can result in detector probes with other Tₘs. In some embodiments, probes can further comprise various modifications such as a minor groove binder (see for example U.S. Pat. No. 6,486,308) to further provide desirable thermodynamic characteristics.

In some embodiments, detection can occur through any of a variety of mobility dependent analytical techniques based on differential rates of migration between different analyte species. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described for example in Published P.C.T. Application WO04/46344 to Rosenblum et al., and WO01/92579 to Wenz et al. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears at al., Nat. Med. 9:14045, including supplements, 2003). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the current teachings.

“Aberration” Means a genomic structural variation or alteration of DNA. Examples include over-/under-expression, copy number amplification/deletion, mutation, gene fusion, and the like.

In one aspect, the disclosure provides a method of treating cancer in a patient in need thereof, comprising obtaining a sample from the patient, analyzing the sample for one or more oncologic variations, determining the patient is amenable to treatment with LNS8801 if one or more oncogenic variations is found, and administering to the patient an effective amount of LNS8801.

In another aspect, the disclosure provides a method of identifying a patient amenable to treatment with LNS8801 therapy, comprising obtaining a sample from the patient, analyzing the sample for one or more oncologic variations, determining the patient is amenable to treatment with LNS8801 If one or more oncogenic variations is found, and administering to the patient an effective amount of LNS8801.

Patient samples include bodily fluid and tissue sample. The samples can be any sample containing polynucleotides or polypeptides of interest and obtained from body fluid (blood, urine, saliva, phlegm, gastric juices, etc.), cultured cells, biopsies, or other tissue preparations, including hard and soft tissues, i.e., tissue that has undergone and not undergone ossification or calcification, respectively.

Analyzing patient samples for oncologic variations can be carried out using methods well-known in the art for elucidating, e.g., sequence, structure, abundance and location of polynucleotides and polypeptides or polypeptides of interest, e.g., in situ hybridization (fluorescence or otherwise), PCR, nucleic acid sequencing, immunohistochemistry, and the like.

Oncologic variations can be effected in various ways including gene fusions, gene mutations, gene duplications and combinations thereof on, e.g., oncogenes, tumor suppressor genes and DNA repair genes.

In embodiments comprising one or more gene fusions, the fusion(s) can include translocations, interstitial deletions, and chromosomal inversions.

In some embodiments, the gene fusion includes DNA from an oncogene and DNA from a second gene, and the oncogene can include a growth factor, a receptor tyrosine kinase, a cytoplasmic tyrosine kinase, a cytoplasmic serine/threonine kinase and regulatory subunit(s) thereof, a regulatory GTPase, or a transcription factor.

In some gene fusions that include a growth factor, the growth factor includes Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF). Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF). Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2). Interleukins, Keratinocyte growth factor (KGF), Migration-stimulating factor (MSF). Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor. T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

In some gene fusions that include a receptor tyrosine kinase, the receptor tyrosine kinase Includes ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu, and FGFR.

In some gene fusions that include a cytoplasmic tyrosine kinase, the cytoplasmic tyrosine kinase includes a member of the SRC family, the Syk-ZAP-70 family, the BTK family, JAK, and Abl.

In some gene fusions that include a cytoplasmic serine/threonine kinase and/or regulatory subunit(s) thereof, the cytoplasmic serine/threonine kinase and/or regulatory subunit(s) thereof includes Raf kinase, MEK, MAPK, and cyclin dependent kinases (e.g., CDK4 and CDK8).

In some gene fusions that include a regulatory GTPase, the regulatory GTPase includes a member of the RAS family, e.g., KRas, NRas, HRas.

In some gene fusions that include a transcription factor, the transcription factor includes a member of the MYC family, e.g., c-myc, l-myc, n-myc.

In some gene fusions that include an oncogene, the oncogene includes Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), interleukin-6 (IL-8), Macrophage colony-stimulating factor (M-CSF), Granulocyte colony-stimulating factor (G-CSF), Granulocyte macrophage colony-stimulating factor (GM-CSF). Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF). Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2). Interleukins, Keratinocyte growth factor (KGF), Migration-stimulating factor (MSF). Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor. T-cell growth factor (TCGF). Thrombopoietin (TPO), Transforming growth factor alpha (TGF-α), Transforming growth factor beta (TGF-β), Tumor necrosis factor-alpha (TNF-α), Vascular endothelial growth factor (VEGF), WNT, ALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR1OP, NFKB2, BCL11A, FGFR2, NRAS, BCL2, FUS, NTRK1, BCL3, GOLGA5, NUP214, BCL6, GOPC, PAX8, BCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

In some embodiments, the gene fusion includes DNA from a tumor suppressor gene and DNA from a second gene, which tumor suppressor gene can include a caretake gene (e.g., BRCA1, BRCA2) or a gatekeeper gene (e.g., P53, NPM1, TPS3, RB, CDKN2A, CDKN2B, P21).

In some gene fusions that include a tumor suppressor gene, the tumor suppressor gene includes APC, IL2, TNFAIP3, ARHGEF12 JAK2, TP53 (P53), ATM, MAP2K1-MAP2K3, MAP2K4, MAP2K5-MAP2K7, TSC1, BCL11B, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SUZ12, GPC3, SYK, IDH1, and TCF3.

In some embodiments the gene fusion comprises DNA from a DNA repair gene and DNA from a second gene, which DNA repair gene codes for a protein that is involved in, e.g., homologous recombination, non-homologous end joining, single-strand annealing, base excision repair, nucleotide excision repair and mismatch repair.

In gene fusions that include DNA repair genes that code for proteins involved in homologous recombination, the DNA repair genes include ATM, ATR, PAXIP, RPA, BRCA1, BRCA2, RAD51, RFC, ERCC1, and MSH3. In gene fusions that Include DNA repair genes that code for a protein involved in non-homologous end joining, the DNA repair genes include ATM, ATR, PAXIP, and PARP1. In gene fusions that include DNA repair genes that code for a protein involved in single-strand annealing, the DNA repair genes include ATM, ATR, RPA, ERCC1, and MSH3. In gene fusions that Include DNA repair genes that code for a protein Involved in base excision repair, the DNA repair genes include RFC, XRCC1, PCNA, and PARP1. In gene fusions that include DNA repair genes that code for a protein involved in nucleotide excision repair, the DNA repair genes include RPA, RFC, XRCC1, PCNA, and ERCC1. In gene fusions that include DNA repair genes that code for a protein involved in mismatch repair, the DNA repair genes include PCNA, MSH3, MSH2, MLH1, PMS1, PMS2, and MSH6.

In some embodiments, the DNA repair gene includes ATM, ATR, PAXIP, RPA, BRCA1, BRCA2, RAD51, RFC, XRCC1, PCNA, PARP1, ERCC1, MSH3, MSH2, MLH1, PMS1, PMS2, MSH6, and in some embodiments the DNA repair gene is selected from the group consisting of ATM, ATR, PAXIP, RPA, BRCA1, BRCA2, RAD51, RFC, XRCC1, PCNA, PARP1, ERCC1, MSH3, MSH2, MLH1. PMS1, PMS2, and MSH6.

In some embodiments, the gene fusion is an ALK fusion, which, in embodiments, upregulates anaplastic lymphoma kinase (ALK) activity. In embodiments, the ALK fusion is selected from the group consisting of NPM-ALK, ALO17-ALK, TFG-ALK, MSN-ALK, TPM3-ALK, TPM4-ALK, ATIC-ALK, MYH9-ALK, CLTC-ALK, TRAF1-ALK, EML4-ALK, KIF5B-ALK, TFG-ALK, KLC1-ALK, PTPN3-ALK, HIP1-ALK, TPR-ALK, STRN-ALK, SEC31A-ALK, RANBP2-ALK, PPFIBP1-ALK, CARS-ALK, SQSTM1-ALK, SEC31A-ALK, VCL-ALK, C2orf44-ALK, FN1-ALK, GFPT1-ALK, and TFG-ALK.

In some ALK fusions, the activity and/or amount of one or more proteins from the Myc family genes, e.g., c-myc, l-myc, and n-myc, are upregulated. MYC is known to be involved broadly in many cancers, and its expression is estimated to be elevated or deregulated in up to 70% of human cancers.

Oncogenic variations can also occur through mutations of the genetic code. In the gene mutation-related embodiments described herein, mutations include one or more base substitutions, deletions, insertions, or combinations thereof.

In some embodiments, the gene mutation(s) include DNA from an oncogene, which oncogenes include growth factors, receptor tyrosine kinases, cytoplasmic tyrosine kinases, cytoplasmic serine/threonine kinases and regulatory subunit(s) thereof, regulatory GTPases, and transcription factors.

In some gene mutations that include DNA from a growth factor gene, the growth factor gene includes Adrenomedullin (AM). Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

In some gene mutations that include DNA from a receptor tyrosine kinase gene, the receptor tyrosine kinase includes ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu, and FGFR.

In some gene mutations that include DNA from a cytoplasmic tyrosine kinase gene, the cytoplasmic tyrosine kinase includes one or more members of the of the SRC family, the Syk-ZAP-70 family, the BTK family, JAK and Abl.

In some gene mutations that include DNA from a cytoplasmic serine/threonine kinase gene, the cytoplasmic serine/threonine kinase and regulatory subunit(s) thereof includes Raf kinase, MEK, and cyclin dependent kinases, e.g., CDK4, CDK8.

In some gene mutations that include DNA from a regulatory GTPase gene, the regulatory GTPase includes one or more members of the RAS family, e.g., KRas, NRas, and HRas.

In some gene mutations that include DNA from a transcription factor gene, the transcription factor includes one or more of members of the MYC family, e.g., c-myc, l-myc, n-myc.

In some gene mutations that include DNA from an oncogene gene, the oncogene is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP). Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNTALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR10P, NFKB2, BCL11A, FGFR2, NRAS, BCL2, FUS, NTRK1, BCL3, GOLGA5, NUP214, BCL6, GOPC, PAX8, BCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

In some embodiments, the gene mutation(s) include DNA from a tumor suppressor gene, which tumor suppressor gene can include a caretaker gene (e.g., BRCA1, BRCA2) or a gatekeeper gene (e.g., P53, NPM1, TP53, RB, CDKN2A, CDKN2B, P21).

In some embodiments, the one or more mutations occurs in a tumor suppressor gene selected from the group consisting of APC, IL2, TNFAIP3, ARHGEF12 JAK2, TP53 (P53), ATM, MAP2K1-MAP2K3, MAP2K4, MAP2K5-MAP2K7, TSC1, BCL11B. MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PAL82, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SU212, GPC3, SYK, IDH1, and TCF3.

In some embodiments, the gene mutation(s) include DNA from a DNA repair gene. The DNA repair gene can code for a protein that is involved in, e.g., homologous recombination, non-homologous end joining, single-strand annealing, base excision repair, nucleotide excision repair or mismatch repair.

In some gene mutations that include DNA from a DNA repair gene, the DNA repair gene codes for a protein that is involved in homologous recombination, including ATM, ATR, PAXIP, RPA, BRCA1, BRCA2, RAD51, RFC, ERCC1, and MSH3. In some gene mutations that include DNA from a DNA repair gene, the DNA repair gene codes for a protein that is involved in non-homologous end joining, e.g., ATM, ATR, PAXIP, and PARP1. In some gene mutations that include DNA from a DNA repair gene, the DNA repair gene codes for a protein that is involved in single-strand annealing, e.g., ATM, ATR, RPA, ERCC1, and MSH3. In some gene mutations that include DNA from a DNA repair gene, the DNA repair gene codes for a protein that is involved in base excision repair, e.g., RFC, XRCC1, PCNA, and PARP1. In some gene mutations that include DNA from a DNA repair gene, the DNA repair gene codes for a protein that is involved in nucleotide excision repair, e.g., RPA, RFC, XRCC1, PCNA, and ERCC1. In some gene mutations that include DNA from a DNA repair gene, the DNA repair gene codes for a protein that is involved in mismatch repair, e.g., PCNA, MSH3, MSH2, MLH1, PMS1, PMS2, and MSH6. In some gene mutations that include DNA from a DNA repair gene, the DNA repair gene includes ATM, ATR, PAXIP, RPA, BRCA1, BRCA2, RAD51, RFC, XRCC1, PCNA, PARP1, ERCC1, MSH3, MSH2, MLH1, PMS1, PMS2, and MSH6.

In some embodiments the oncogenic variation comprises one or more DNA mutations in the ALK gene. In some embodiments, the ALK mutation(s) upregulates ALK activity. In some embodiments, the one or more ALK mutations is selected from the group consisting of P496L, P542R, S631I, V1135E, C1156Y and L1196M. In some embodiments, the one or more ALK mutations upregulates the activity and/or amount of one or more proteins from the Myc family genes, e.g., c-myc, l-myc, n-myc.

Gene amplification is an increase in the number of copies of a gene that may also be accompanied by an increase in the RNA and protein made from that gene. Gene amplification is common in cancer cells, and some amplified genes may cause cancer cells to grow or become resistant to anticancer drugs.

In some embodiments the oncogenic variation comprises one or more DNA amplifications, which, in some embodiments, occurs in an oncogene. In some gene amplifications, the oncogene that undergoes amplification comprises a growth factor, a receptor tyrosine kinase, a cytoplasmic tyrosine kinase, a cytoplasmic serine/threonine kinase and regulatory subunit(s) thereof, a regulatory GTPase, or a transcription factor.

In some gene amplifications wherein the amplified oncogene is a growth factor, the growth factor is selected from the group consisting of Adrenomedullin (AM). Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-8 (IL-8), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1). Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

In some gene amplifications, the amplified oncogene is a receptor tyrosine kinase, e.g., ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu and FGFR. In some gene amplifications, the amplified oncogene is a cytoplasmic tyrosine kinase, e.g., a member of the SRC family, the Syk-ZAP-70 family, the BTK family, JAK and Abl. In some gene amplifications, the amplified oncogene is a cytoplasmic serine/threonine kinase, e.g., Raf kinase, MEK, and cyclin dependent kinases, e.g., CDK4, CDK8. In some gene amplifications, the amplified oncogene is a regulatory GTPase, e.g., a member of the RAS family, e.g., KRas, NRas and HRas. In some gene amplifications, the amplified oncogene is a transcription factor, e.g., a member of the MYC family, e.g., c-myc, l-myc and n-myc.

In some embodiments the amplified oncogene is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-8), Macrophage colony-stimulating factor (M-CSF), Granulocyte colony-stimulating factor (G-CSF). Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF). Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNTALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR1OP, NFKB2, BCL11A, FGFR2, NRAS, BCL2, FUS, NTRK1, BCL3, GOLGA5, NUP214, BCL6, GOPC, PAX8, BCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

In some embodiments the oncogenic variation comprises one or more DNA amplifications, which, in some embodiments, occurs in a tumor suppressor gene. In some gene amplifications, the tumor suppressor gene comprises a caretake gene or a gatekeeper gene. In some embodiments, the caretaker gene comprises BRCA1 or BRCA2, and in some embodiments the gatekeeper gene comprises P53, NPM1, TP53, RB, CDKN2A, CDKN2B or P21.

In some embodiments the tumor suppressor gene is selected from the group consisting of APC, IL2, TNFAIP3, ARHGEF12 JAK2, TP53 (P53), ATM, MAP2K1-MAP2K3, MAP2K4, MAP2K5-MAP2K7, TSC1, BCL11B, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SUZ12, GPC3, SYK, IDH1 and TCF3.

In some embodiments the oncogenic variation comprises one or more DNA amplifications, which, in some embodiments, occurs in a DNA repair gene. DNA repair genes code for a proteins that are involved in processes such as homologous recombination, non-homologous end joining, single-strand annealing, base excision repair, nucleotide excision repair or mismatch repair. DNA repair genes that code for proteins that are involved in homologous recombination include ATM, ATR, PAXIP, RPA, BRCA1, BRCA2, RAD51, RFC, ERCC1, and MSH3. DNA repair genes that code for proteins that are involved in non-homologous end joining include ATM. ATR, PAXIP, and PARP1. DNA repair genes that code for proteins that are involved in single-strand annealing processes include ATM, ATR, RPA, ERCC1, and MSH3. DNA repair genes that code for proteins that are involved in base excision repair include RFC, XRCC1, PCNA, and PARP1. DNA repair genes that code for proteins that are involved in nucleotide excision repair include RPA, RFC, XRCC1, PCNA and ERCC1. DNA repair genes that code for proteins that are involved in in mismatch repair include PCNA, MSH3, MSH2, MLH1, PMS1, PMS2, and MSH6.

In some gene amplifications that include DNA repair genes, the DNA repair gene includes ATM, ATR. PAXIP, RPA, BRCA1, BRCA2, RAD51, RFC, XRCC1, PCNA, PARP1, ERCC1, MSH3, MSH2, MLH1, PMS1, PMS2, and MSH6.

In some embodiments the oncogenic variation comprises one or more DNA amplifications in the ALK gene, which amplification can upregulate ALK abundance and/or activity and, in embodiments, can upregulate the activity and/or amount of one or more proteins from the Myc family genes, e.g., c-myc, n-myc or l-myc.

In some embodiments the oncogenic variation comprises one or more DNA amplifications in a MET family gene, including c-MET, in the CCND1 gene, in the MDM2 gene, in the ERBB2 gene, in the EGFR gene, in the KRAS gene, in the NRAS gene, in the HRAS gene, in the BRAF gene, in the c-KIT gene, in the p53 gene, in the NOTCH gene, in the STK11gene, in the NF1gene, in the ATM gene, in the PI3K gene, or in the MEK gene.

Antineoplastic resistance is the ability of cancer cells to survive and grow despite exposure to anti-cancer therapies. Some oncogenic variations, such as gene fusions, mutations and amplifications described herein, can confer such resistance. In some embodiments the one or more oncogenic variations confers resistance to one or more cancer therapies. Resistance can be conferred via different oncogenic pathways, and in some embodiments, the resistance is driven by an increase in amount or activity of one or more proteins from Myc family genes, e.g., c-myc, n-myc or l-myc.

In some embodiments, the one or more cancer therapies comprises immune checkpoint therapy agents directed against, e.g., PD-1, PD-L1, CTLA4. CD40, OX40, TIGIT, CD137, or targeted inhibitors against, e.g., EGFR, BRAF, MEK, ALK, JAK1/2, VEGF, SRC, BTK, AKT, MTOR, BCL-2, ESR1, FGFR, MET, or combinations thereof.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

In this disclosure, “comprises,” “comprising,” “containing,” and “having” and the like can have the meaning ascribed to them in U.S. Patent law and can mean “includes,” “including,” and the like. “Consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. By contrast, the transitional phrase “consisting of” excludes any element, step, or ingredient not specified.

As used in the description herein and throughout the claims that follow, the meaning of “a,” “an,” and “the” includes plural reference unless the context clearly dictates otherwise.

It is understood that the examples and embodiments described herein are for illustrative purposes only and that various modifications or changes in light thereof will be suggested to persons skilled in the art and are to be Included within the spirit and purview of this application and scope of the appended claims. All publications, patents, and patent applications cited herein are hereby incorporated by reference in their entirety for all purposes.

### EXAMPLES

### Example 1

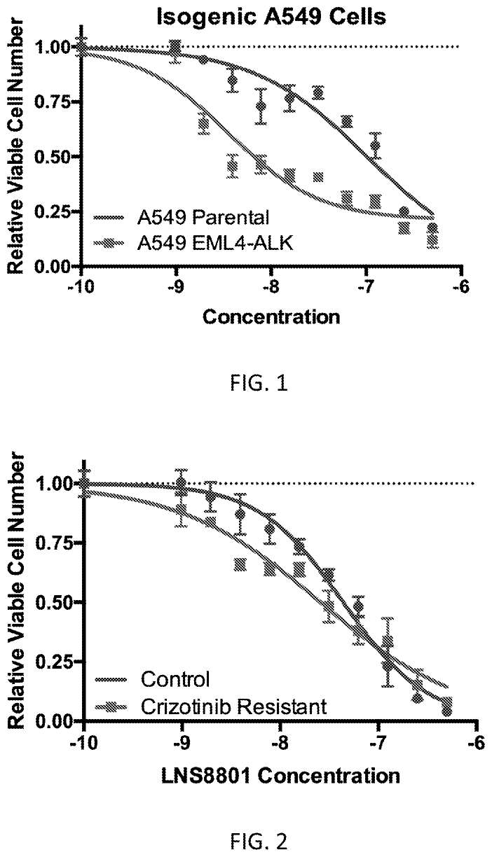

Parental cells and EML4-ALK A549 NSCLC cells (5,000 cells) were plated in a 96-well tissue culture plate. Cells were treated with an array of ½ dilutions of LNS8801 for 4 days with 4 technical replicates for each condition (500 nM, 250 nM, 125 nM, 62.5 nM, 31.25 nM, 15.83 nM, 7.81 nM, 3.91M, 1.95 nM, 0.98 nM and 0 nM). After 4 days, relative viable cells were determined by Incubating cells with CCK-8 proliferation dye for 1 hour and measuring absorbance at 450 nM. Relative cell numbers were calculated by subtracting baseline absorbance of the media followed by normalization to the 0 nM treated control wells. Results are shown in FIG. 1. LNS8801 effectively reduced EML4-ALK A549 cell viability over control A549 cells.

### Example 2

Crizotinib naive and resistant EML4-ALK A549 NSCLC cells (5,000 cells) were plated in a 98-well tissue culture plate. Crizotinib resistant cells were generated by prolonged culturing of EML4-ALK A549 cells under increasing concentrations of Crizotinib over 6 weeks. Cells were treated with an array of % dilutions of LNS8801 for 4 days with 4 technical replicates for each condition (500 nM, 250 nM, 125 nM, 62.5 nM, 31.25 nM, 15.63 nM, 7.81 nM, 3.91 nM, 1.95 nM, 0.98 nM and 0 nM). After 4 days, relative viable cells were determined by incubating cells with CCK-8 proliferation dye for 1 hour, and measuring absorbance at 450 nM. Relative cell numbers were calculated by subtracting baseline absorbance of the media followed by normalization to the 0 nM treated control wells. Results are shown in FIG. 2.

### Example 3

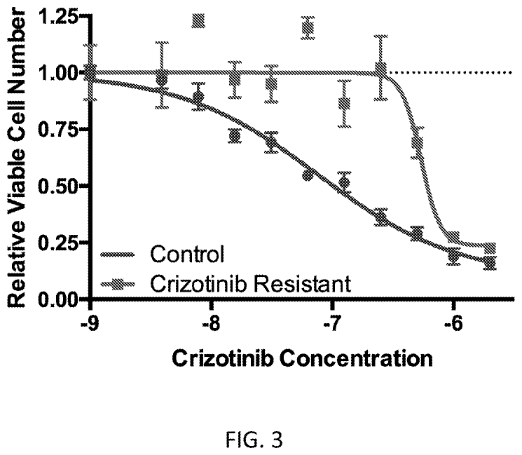

Crizotinib naive and resistant EML4-ALK A549 NSCLC cells (5,000 cells) were plated in a 96-well tissue culture plate. Crizotinib resistant cells were generated by prolonged culturing of EML4-ALK A549 cells under increasing concentrations of Crizotinib over 6 weeks. Cells were treated with an array of ½ dilutions of Crizotinib for 4 days with 4 technical replicates for each condition (2000 nM, 1000 nM, 500 nM, 250 nM, 125 nM, 82.5 nM, 31.25 nM, 15.83 nM, 7.81 nM, 3.91 nM, 1.95 nM, 0.98 nM and 0 nM Crizotinib). After 4 days, relative viable cells were determined by incubating cells with CCK-8 proliferation dye for 1 hour, and measuring absorbance at 450 nM. Relative cell numbers were calculated by subtracting baseline absorbance of the media followed by normalization to the 0 nM treated control wells. Results are shown in FIG. 3.

Crizotinib appeared unable to reduce the relative viability of the Crizotinib-resistant cells, which cells remained essentially 100% viable to 250 nM, as compared to controls. In contrast, LNS8801 was able to effectively reduce viability of Crizotinib-resistant cells (Example 2 and FIG. 2).

EML4-ALK fusions can result in constitutive activation of ALK, which can lead to the activation of oncogenic signaling through multiple pathways via ALK-interacting or ALK-regulated partners such as PI3K/Akt, JAK/STAT, RAS/RAF/MEK/ERK and MYC. Not to be bound by theory, but LNS8801, a G protein-coupled estrogen receptor (GPER) agonist, may, among other activities, indirectly downregulate MYC (e.g., through one or more other proteins, like protein kinase A, PKA), in opposition to the activity of EML4-ALK. MYC is a regulator of numerous genes implicated in cancers and is often overexpressed in cancers. Thus, LNS8801 may be an effective agent against MYC-dependent cancers caused by many of the oncologic variations described herein.

Additional aspects and embodiments of the disclosure are provided by the claims below, which can be combined in any number and in any combination not technically or logically inconsistent.

1. A method of treating cancer in a patient in need thereof, comprising:
obtaining a sample from the patient;
analyzing the sample for one or more oncologic variations;
determining the patient is amenable to treatment with LNS8801 if one or more oncogenic variations is found; and
administering to the patient an effective amount of LNS8801.

2. The method of claim 1, wherein the sample comprises a bodily fluid or tissue sample.

3. The method of claim 2, wherein the bodily fluid comprises one or more of blood, serum, plasma, saliva, oral swab (e.g., cheek swab), cerebrospinal fluid (CSF), or mucosal secretion.

4. The method of claim 3, wherein the bodily fluid is blood or saliva.

5. The method of claim 2, wherein the tissue sample comprises one or more of soft tissue or hard tissue.

6. The method of claim 5, wherein the soft tissue comprises a bodily tissue that has not undergone ossification or calcification.

7. The method of claim 1, wherein the analyzing the sample for one or more oncologic variations comprises hybridization (e.g., in situ hybridization, microarrays, fluorescent barcodes), PCR (e.g., PCR, quantitative PCR, amplification-refractory mutation system (ARMS), blocker PCR, digital PCR), nucleic acid sequencing, immunohistochemistry, or electrophoresis.

8. The method of claim 1, wherein the oncologic variations comprise one or more gene fusions, gene mutations, gene duplications or combinations thereof.

9. The method of claim 2, wherein the oncologic variation is a gene fusion.

10. The method of claim 9, wherein the gene fusion comprises DNA from an oncogene and DNA from a second gene.

11. The method of claim 10, wherein the oncogene comprises a growth factor, a receptor tyrosine kinase, a cytoplasmic tyrosine kinase, a cytoplasmic serine/threonine kinase and regulatory subunit(s) thereof, a regulatory GTPase, or a transcription factor.

12. The method of claim 11, wherein the growth factor is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

13. The method of claim 11, wherein the receptor tyrosine kinase is selected from the group consisting of ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu, and FGFR.

14. The method of claim 11, wherein the cytoplasmic tyrosine kinase is selected from the group consisting of a member of the SRC family, the Syk-ZAP-70 family, the BTK family, JAK and Abl.

15. The method of claim 11, wherein the cytoplasmic serine/threonine kinase and regulatory subunit(s) thereof is selected from the group consisting of Raf kinase, MEK, MAPK, and cyclin dependent kinases CDK4, and CDK8.

16. The method of claim 11, wherein the regulatory GTPase is selected from the group consisting of a member of the RAS family (KRas, NRas and HRas).

17. The method of claim 11, wherein the transcription factor is selected from the group consisting of a member of the MYC family (c-myc, l-myc, and n-myc).

18. The method of claim 11, wherein the oncogene is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), WNT, ALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR1OP, NFKB2, BCL11A, FGFR2, NRAS, BCL2, FUS, NTRK1, BCL3, GOLGA5, NUP214, BCL6, GOPC, PAX8, BCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

19. The method of claim 9, wherein the gene fusion comprises DNA from a tumor suppressor gene and DNA from a second gene.

20. The method of claim 19, wherein the tumor suppressor gene comprises a caretaker gene or a gatekeeper gene.

21. The method of claim 20, wherein the caretaker gene comprises BRCA1 or BRCA2.

22. The method of claim 20, wherein the gatekeeper gene comprises P53 and NPM1.

23. The method of claim 19, wherein the tumor suppressor gene is selected from the group consisting of APC, IL2, TNFAIP3, ARHGEF12 JAK2, TP53 (P53), ATM, MAP2K1-MAP2K3, MAP2K4, MAP2K5-MAP2K7, TSC1, BCL11B, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SUZ12, GPC3, SYK, IDH1, and TCF3.

24. The method of claim 9, wherein the gene fusion comprises DNA from a DNA repair gene and DNA from a second gene.

25. The method of claim 24, wherein the DNA repair gene codes for a protein that is involved in homologous recombination, non-homologous end joining, single-strand annealing, base excision repair, nucleotide excision repair or mismatch repair.

26. The method of claim 25, wherein the DNA repair gene codes for a protein that is involved in homologous recombination.

27. The method of claim 26, wherein the DNA repair gene that codes for a protein that is involved in homologous recombination comprises ATM, ATR, PAXIP, RPA, BRCA1, BRCA2, RAD51, RFC, ERCC1 or MSH3.

28. The method of claim 25, wherein the DNA repair gene codes for a protein that is involved in non-homologous end joining.

29. The method of claim 28, wherein the DNA repair gene that codes for a protein that is involved in non-homologous end joining comprises ATM, ATR, PAXIP or PARP1.

30. The method of claim 25, wherein the DNA repair gene codes for a protein that is involved in single-strand annealing.

31. The method of claim 30, wherein the DNA repair gene that codes for a protein that is involved in single-strand annealing comprises ATM, ATR, RPA, ERCC1 or MSH3.

32. The method of claim 25, wherein the DNA repair gene codes for a protein that is involved in base excision repair.

33. The method of claim 32, wherein the DNA repair gene that codes for a protein that is involved in base excision repair comprises RFC, XRCC1, PCNA or PARP1.

34. The method of claim 25, wherein the DNA repair gene codes for a protein that is Involved in nucleotide excision repair.

35. The method of claim 34, wherein the DNA repair gene that codes for a protein that is involved in nucleotide excision repair comprises RPA, RFC, XRCC1, PCNA or ERCC1.

36. The method of claim 25, wherein the DNA repair gene codes for a protein that is involved in mismatch repair.

37. The method of claim 36, wherein the DNA repair gene that codes for a protein that is involved in mismatch repair comprises PCNA, MSH3, MSH2, MLH1, PMS1, PMS2 or MSH6.

38. The method of claim 24, wherein the DNA repair gene comprises ATM, ATR, PAXIP, RPA, BRCA1, BRCA2, RAD51, RFC, XRCC1, PCNA, PARP1, ERCC1, MSH3, MSH2, MLH1, PMS1, PMS2 or MSH6.

39. The method of claim 24, wherein the DNA repair gene is selected from the group consisting of ATM, ATR, PAXIP, RPA, BRCA1, BRCA2, RAD51, RFC, XRCC1, PCNA, PARP1, ERCC1, MSH3, MSH2, MLH1, PMS1, PMS2 and MSH6.

40. The method of claim 9, wherein the gene fusion is an ALK fusion.

41. The method of claim 40, wherein the ALK fusion upregulates ALK activity.

42. The method of claim either one of claim 40 or 41, wherein the ALK fusion is selected from the group consisting of NPM-ALK, ALO17-ALK, TFG-ALK, MSN-ALK, TPM3-ALK, TPM1-ALK, TPM4-ALK, ATIC-ALK, MYH9-ALK, CLTC-ALK, TRAF1-ALK, EML4-ALK, KIF5B-ALK, TFG-ALK, KLC1-ALK, PTPN3-ALK, HIP1-ALK, TPR-ALK, STRN-ALK, SEC31A-ALK, RANBP2-ALK, PPFIBP1-ALK, CARS-ALK, SQSTM1-ALK, SEC31A-ALK, VCL-ALK, C2orf44-ALK, FN1-ALK, GFPT1-ALK, and TFG-ALK.

43. The method of any one of claims 1-42, wherein the gene fusion upregulates the activity and/or amount of one or more proteins from the Myc family genes.

44. The method of any one of claims 1-42, wherein the gene fusion comprises a translocation, an interstitial deletion, or a chromosomal inversion.

45. The method of any one of claims 1-8, wherein the oncogenic variation comprises one or more DNA mutations.

46. The method of claim 45, wherein the one or more mutations occurs in an oncogene comprising a growth factor, a receptor tyrosine kinase, a cytoplasmic tyrosine kinase, a cytoplasmic serine/threonine kinase and regulatory subunit(s) thereof, a regulatory GTPase, or a transcription factor.

47. The method of claim 46, wherein the growth factor is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-8 (IL-8), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

48. The method of claim 46, wherein the receptor tyrosine kinase is selected from the group consisting of ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu and FGFR.

49. The method of claim 46, wherein the cytoplasmic tyrosine kinase is selected from the group consisting of a member of the SRC family, the Syk-ZAP-70 family, the BTK family, JAK and Abl.

50. The method of claim 48, wherein the cytoplasmic serine/threonine kinase and regulatory subunit(s) thereof is selected from the group consisting of Raf kinase, MEK, and cyclin dependent kinases CDK4 and CDK8.

51. The method of claim 48, wherein the regulatory GTPase is selected from the group consisting of a member of the RAS family (e.g., KRas, NRas, HRas).

52. The method of claim 48, wherein the transcription factor is selected from the group consisting of a member of the MYC family (e.g., c-myc, l-myc, n-myc).

53. The method of claim 46, wherein the one or more mutations occurs in an oncogene selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), insulin-like growth factor-2 (IGF-2), interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), WNT, ALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR1OP, NFKB2, BCL11A, FGFR2, NRAS, BCL2, FUS, NTRK1, BCL3, GOLGA5, NUP214, BCL6, GOPC, PAX8, BCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

54. The method of claim 45, wherein the one or more mutations occurs in a tumor suppressor gene.

55. The method of claim 54, wherein the tumor suppressor gene comprises a caretaker gene or a gatekeeper gene.

56. The method of claim 55, wherein the caretaker gene comprises BRCA1 or BRCA2.

57. The method of claim 55, wherein the gatekeeper gene comprises P53, NPM1, TP53, RB, CDKN2A, CDKN2B or P21.

58. The method of claim 56 54, wherein the tumor suppressor gene is selected from the group consisting of APC, IL2, TNFAIP3, ARHGEF12 JAK2, TPS3 (P53), ATM, MAP2K1-MAP2K3, MAP2K4, MAP2K5-MAP2K7, TSC1, BCL118, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SUZ12, GPC3, SYK, IDH1 and TCF3.

59. The method of claim 45, wherein the one or more mutations occurs in a DNA repair gene.

60. The method of claim 59, wherein the DNA repair gene codes for a protein that is involved in homologous recombination, non-homologous end joining, single-strand annealing, base excision repair, nucleotide excision repair or mismatch repair.

61. The method of claim 60, wherein the DNA repair gene codes for a protein that is involved in homologous recombination.

62. The method of claim 61, wherein the DNA repair gene that codes for a protein that is involved in homologous recombination comprises ATM, ATR, PAXIP, RPA, BRCA1, BRCA2, RAD51, RFC, ERCC1 or MSH3.

63. The method of claim 60, wherein the DNA repair gene codes for a protein that is involved in non-homologous end joining.

64. The method of claim 63, wherein the DNA repair gene that codes for a protein that is involved in non-homologous end joining comprises ATM, ATR, PAXIP or PARP1.

65. The method of claim 60, wherein the DNA repair gene codes for a protein that is involved in single-strand annealing.

66. The method of claim 65, wherein the DNA repair gene that codes for a protein that is involved in single-strand annealing comprises ATM, ATR, RPA, ERCC1 or MSH3.

67. The method of claim 60, wherein the DNA repair gene codes for a protein that is involved in base excision repair.

68. The method of claim 67, wherein the DNA repair gene that codes for a protein that is involved in base excision repair comprises RFC, XRCC1, PCNA, or PARP1.

69. The method of claim 60, wherein the DNA repair gene codes for a protein that is involved in nucleotide excision repair.

70. The method of claim 69, wherein the DNA repair gene that codes for a protein that is involved in nucleotide excision repair comprises RPA, RFC, XRCC1, PCNA or ERCC1.

71. The method of claim 60, wherein the DNA repair gene codes for a protein that is involved in mismatch repair.

72. The method of claim 71, wherein the DNA repair gene that codes for a protein that is involved in mismatch repair comprises PCNA, MSH3, MSH2, MLH1, PMS1, PMS2 or MSH6.

73. The method of claim 59, wherein the DNA repair gene comprises ATM, ATR, PAXIP, RPA, BRCA1, BRCA2, RAD51, RFC, XRCC1, PCNA, PARP1, ERCC1, MSH3, MSH2, MLH1, PMS1, PMS2 or MSH6.

74. The method of claim 59, wherein the DNA repair gene is selected from the group consisting of ATM, ATR, PAXIP, RPA, BRCA1, BRCA2, RAD51, RFC, XRCC1, PCNA, PARP1, ERCC1, MSH3, MSH2, MLH1, PMS1, PMS2 and MSH6.

75. The method of claim 45, wherein the one or more mutations occurs in the ALK gene.

76. The method of claim 75, wherein the one or more ALK mutations upregulates ALK activity.

77. The method of claim either one of claim 75 or 76, wherein the one or more ALK mutations is selected from the group consisting of P496L, P542R, S631I, V1135E, C1156Y and L1196M.

78. The method of any one of claims 45-77, wherein the one or more ALK mutations upregulates the activity and/or amount of one or more proteins from the Myc family genes.

79. The method of any one of claims 45-78, wherein the mutation comprises one or more base substitutions, deletions, insertions, or combinations thereof.

80. The method of any one of claims 1-8, wherein the oncogenic variation comprises one or more DNA amplifications.

81. The method of claim 80, wherein the amplification occurs in an oncogene comprising a growth factor, a receptor tyrosine kinase, a cytoplasmic tyrosine kinase, a cytoplasmic serine/threonine kinase and regulatory subunit(s) thereof, a regulatory GTPase, or a transcription factor.

82. The method of claim 81, wherein the growth factor is selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia inhibitory factor (LIF), Interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), Interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), and WNT.

83. The method of claim 81, wherein the receptor tyrosine kinase is selected from the group consisting of ALK, ROS1, ABL, RET, C-KIT, PI3K, epidermal growth factor receptor (EGFR), platelet-derived growth factor receptor (PDGFR), and vascular endothelial growth factor receptor (VEGFR), HER2/neu and FGFR.

84. The method of claim 81, wherein the cytoplasmic tyrosine kinase is selected from the group consisting of a member of the SRC family, the Syk-ZAP-70 family, the BTK family, JAK, and Abl.

85. The method of claim 81, wherein the cytoplasmic serine/threonine kinase and regulatory subunit(s) thereof is selected from the group consisting of Raf kinase, MEK, and cyclin dependent kinases CDK4 and CDK8.

86. The method of claim 81, wherein the regulatory GTPase is selected from the group consisting of a member of the RAS family (e.g., KRas, NRas, HRas).

87. The method of claim 81, wherein the transcription factor is selected from the group consisting of a member of the MYC family (e.g., c-myc, l-myc, n-myc).

88. The method of claim 81, wherein the amplification occurs in an oncogene selected from the group consisting of Adrenomedullin (AM), Angiopoietin (Ang), Autocrine motility factor, Bone morphogenetic proteins (BMPs), Ciliary neurotrophic factor family, Ciliary neurotrophic factor (CNTF), Leukemia Inhibitory factor (LIF), interleukin-6 (IL-6), Macrophage colony-stimulating factor (M-CSF), Granulocyte colony-stimulating factor (G-CSF), Granulocyte macrophage colony-stimulating factor (GM-CSF), Epidermal growth factor (EGF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Glial cell line-derived neurotrophic factor (GDNF), Neurturin, Persephin, Artemin, Growth differentiation factor-9 (GDF9), Hepatocyte growth factor (HGF), Hepatoma-derived growth factor (HDGF), Insulin, Insulin-like growth factor-1 (IGF-1), Insulin-like growth factor-2 (IGF-2), interleukins, Keratinocyte growth factor (KGF), Migration-stimulating factor (MSF), Macrophage-stimulating protein (MSP), also known as hepatocyte growth factor-like protein (HGFLP), Myostatin (GDF-8), Neuregulin 1 (NRG1), Neuregulin 2 (NRG2), Neuregulin 3 (NRG3), Neuregulin 4 (NRG4), Brain-derived neurotrophic factor (BDNF), Nerve growth factor (NGF), Neurotrophin-3 (NT-3), Neurotrophin-4 (NT-4), Placental growth factor (PGF), Platelet-derived growth factor (PDGF), Renalase (RNLS)—Anti-apoptotic survival factor, T-cell growth factor (TCGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), WNT, ALK, ABL1, CCND1, MDM2, ERBB2, EVI1, MYC, ABL2, EWSR1, MYCL/MYCL1, AKT1, FEV, MYCN, AKT2, FGFR1, NCOA4, ATF1, FGFR1OP, NFKB2, BCL11A, FGFR2, NRAS, BCL2, FUS, NTRK1, BCL3, GOLGA5, NUP214, BCL6, GOPC, PAX8, SCR, HMGA1, PDGFB, BRAF, HMGA2, PIK3CA, CARD11, HRAS, PIM1, CBLB, IRF4, PLAG1, CBLC, JUN, PPARG, CCND1, KIT, PTPN11, CCND2, KRAS, RAF1, CCND3, LCK, REL, CDX2, LMO2, RET, CTNNB1, MAF, ROS1, DDB2, MAFB, SMO, DDIT3, MAML2, SS18, DDX6, MDM2, TCL1A, DEK, MET, TET2, EGFR, PDGFR, VEGFR, MITF, TFG, ELK4, MLL, TLX1, ERBB2, MPL, TPR, ETV4, MYB, USP6, and ETV6.

89. The method of claim 80, wherein the amplification occurs in a tumor suppressor gene.

90. The method of claim 89, wherein the tumor suppressor gene comprises a caretaker gene or a gatekeeper gene.

91. The method of claim 90, wherein the caretaker gene comprises BRCA1 or BRCA2.

92. The method of claim 90, wherein the gatekeeper gene comprises P53 or NPM1.

93. The method of claim 89, wherein the tumor suppressor gene is selected from the group consisting of APC, IL2, TNFAIP3, ARHGEF12 JAK2, TPS3 (P53), ATM, MAP2K1-MAP2K3, MAP2K4, MAP2K5-MAP2K7, TSC1, BCL11B, MDM4, TSC2, BLM, MEN1, VHL, BMPR1A, MLH1, WRN, BRCA1, MSH2, WT1, BRCA2, NF1, CARS, NF2, CBFA2T3, NOTCH1, CDH1, NPM1, CDH11, NR4A3, CDK6, NUP98, CDKN2C, PALB2, CEBPA, PML, CHEK2, PTEN, CREB1, RB1, CREBBP, RUNX1, CYLD, SDHB, DDX5, SDHD, EXT1, MARCA4, EXT2, SMARCB1, FBXW7, SOCS1, FH, STK11, FLT3, SUFU, FOXP1, SUZ12, GPC3, SYK, IDH1 and TCF3.

94. The method of claim 80, wherein the amplification occurs in a DNA repair gene.

95. The method of claim 94, wherein the DNA repair gene codes for a protein that is involved in homologous recombination, non-homologous end joining, single-strand annealing, base excision repair, nucleotide excision repair or mismatch repair.

96. The method of claim 95, wherein the DNA repair gene codes for a protein that is involved in homologous recombination.

97. The method of claim 96, wherein the DNA repair gene that codes for a protein that is involved in homologous recombination comprises ATM, ATR, PAXIP, RPA, BRCA1, BRCA2, RAD51, RFC, ERCC1 or MSH3.

98. The method of claim 95, wherein the DNA repair gene codes for a protein that is involved in non-homologous end joining.

99. The method of claim 98, wherein the DNA repair gene that codes for a protein that is involved in non-homologous end joining comprises ATM, ATR, PAXIP, or PARP1.

100. The method of claim 95, wherein the DNA repair gene codes for a protein that is involved in single-strand annealing.

101. The method of claim 100, wherein the DNA repair gene that codes for a protein that is involved in single-strand annealing comprises ATM, ATR, RPA, ERCC1 or MSH3.

102. The method of claim 95, wherein the DNA repair gene codes for a protein that is involved in base excision repair.

103. The method of claim 102, wherein the DNA repair gene that codes for a protein that is involved in base excision repair comprises RFC, XRCC1, PCNA and PARP1.

104. The method of claim 95, wherein the DNA repair gene codes for a protein that is involved in nucleotide excision repair.

105. The method of claim 104, wherein the DNA repair gene that codes for a protein that is involved in nucleotide excision repair comprises RPA, RFC, XRCC1, PCNA and ERCC1.

106. The method of claim 95, wherein the DNA repair gene codes for a protein that is involved in mismatch repair.

107. The method of claim 108, wherein the DNA repair gene that codes for a protein that is Involved in mismatch repair comprises PCNA, MSH3, MSH2, MLH1, PMS1, PMS2 and MSH6.

108. The method of claim 94, wherein the DNA repair gene comprises ATM, ATR, PAXIP, RPA, BRCA1, BRCA2, RAD51, RFC, XRCC1, PCNA, PARP1, ERCC1, MSH3, MSH2, MLH1, PMS1, PMS2 and MSH6.

109. The method of claim 94, wherein the DNA repair gene is selected from the group consisting of ATM, ATR, PAXIP, RPA, BRCA1, BRCA2, RAD51, RFC, XRCC1, PCNA, PARP1, ERCC1, MSH3, MSH2, MLH1, PMS1, PMS2 and MSH6.

110. The method of claim 80, wherein the amplification occurs in the ALK gene.

111. The method of claim 110, wherein the amplification upregulates ALK activity.

112. The method of any one of claims 80-111, wherein the ALK amplification upregulates the activity and/or amount of one or more proteins from the Myc family genes.

113. The method of claim 80, wherein the amplification occurs in a MET family gene.

114. The method of claim 113, wherein the MET family gene is c-MET.

115. The method of claim 80, wherein the amplification occurs in the CCND1 gene.

116. The method of claim 80, wherein the amplification occurs in the MDM2 gene.

117. The method of claim 80, wherein the amplification occurs in the ERBB2 gene.

118. The method of claim 45, wherein the one or more mutations occurs in the EGFR gene.

119. The method of claim 45, wherein the one or more mutations occurs in the KRAS gene.

120. The method of claim 45, wherein the one or more mutations occurs in the NRAS gene.

121. The method of claim 45, wherein the one or more mutations occurs in the HRAS gene.

122. The method of claim 45, wherein the one or more mutations occurs in the BRAF gene.

123. The method of claim 45, wherein the one or more mutations occurs in the c-KIT gene.

124. The method of claim 45, wherein the one or more mutations occurs in the p53 gene.

125. The method of claim 45, wherein the one or more mutations occurs in the NOTCH gene.

126. The method of claim 45, wherein the one or more mutations occurs in the STK11gene.

127. The method of claim 45, wherein the one or more mutations occurs in the NF1gene.

128. The method of claim 45, wherein the one or more mutations occurs in the ATM gene.

129. The method of claim 45, wherein the one or more mutations occurs in the PI3K gene.

130. The method of claim 45, wherein the one or more mutations occurs in the MEK gene.

131. The method of claim 45, wherein the gene fusion is a MYC fusion.

132. The method of claim 45, wherein the gene fusion is a ROS1 fusion.

133. The method of claim 45, wherein the gene fusion is a ABL fusion.

134. The method of claim 45, wherein the gene fusion is a RET fusion.

135. The method of claim 45, wherein the gene fusion is a NPM1 fusion.

136. The method of any one of claims 113-135, wherein the one or more oncologic variations upregulates the activity and/or amount of one or more proteins from the Myc family genes.

137. The method of any one of claims 1-136, wherein the one or more oncogenic variations confers resistance to one or more cancer therapies.

138. The method of claim 137, wherein the resistance is driven by an increase in amount or activity of one or more proteins from Myc family genes.

139. The method of claim 138, wherein the Myc family genes comprise MYC (c-myc), MYCN (n-myc) or MYCL/MYCL1 (l-myc).

140. The method of claim 139, wherein the Myc family genes are selected from the group consisting of MYC (c-myc), MYCN (n-myc) and MYCL (l-myc).

141. The method of any one of claims 137-140, wherein the one or more cancer therapies comprises an immune checkpoint therapy agent directed against PD-1, PD-L1, CTLA4, CD40, OX40, TIGIT, CD137, or combinations thereof.

142. The method of one of claims 137-140, wherein the one or more cancer therapies comprises a targeted inhibitor against EGFR, BRAF, MEK, ALK, JAK1/2, VEGF, SRC, BTK, AKT, MTOR, BCL-2, ESR1, FGFR, MET, or combinations thereof.
